(19) United States
(12) Patent Application Publication
(10) Pub. No.: US 2021/0113809 A1
(43) Pub. Date: Apr. 22, 2021

(54) Rapidly Insertable Central Catheter and Methods Thereof

(71) Applicant: Bard Access Systems, Inc., Salt Lake City, UT (US)

(72) Inventor: Glade H. Howell, Draper, UT (US)

(21) Appl. No.: 17/074,405

(22) Filed: Oct. 19, 2020

Related U.S. Application Data
(60) Provisional application No. 62/923,320, filed on Oct. 18, 2019.

(51) Int. Cl. A61M 25/00

(52) U.S. Cl. CPC A61M 25/0023; A61M 25/0026; A61M 25/0009

(57) ABSTRACT

Rapidly inserted central catheters (“RICC”) and methods thereof are disclosed. For example, a RICC can include a first section in a distal-end portion of a catheter tube, a second section in the distal-end portion of the catheter tube proximal of the first section of the catheter tube, and a junction joining the first and second sections of the catheter tube. The first section of the catheter tube can be formed of a first polymeric material having a first durometer. The second section of the catheter tube can be formed of a second polymeric material having a second durometer less than the first durometer. The first section of the catheter tube can have a proximal-end portion disposed in a receptacle of the junction and solvent bonded thereto. For example, a method can include a method of making or using a RICC.

### CROSS-REFERENCE

This application claims the benefit of priority to U.S. Provisional Patent Application No. 62/923,320 filed Oct. 18, 2019, which is incorporated by reference in its entirety into this application.

### BACKGROUND

A central venous catheter (“CVC”) is formed of a material having a relatively low durometer, which contributes to the CVC having a lack of column strength. Due to the lack of column strength, CVCs are commonly introduced into patients and advanced through vasculatures thereof by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter into a patient and advancing the catheter through a vasculature thereof.

Disclosed herein are rapidly insertable central catheters (“RICCs”) and methods thereof that address the foregoing.

### SUMMARY

Disclosed herein is a RICC including, in some embodiments, a first section in a distal-end portion of a catheter tube, a second section in the distal-end portion of the catheter tube proximal of the first section of the catheter tube, and a junction joining the first and second sections of the catheter tube. The first section of the catheter tube is formed of a first polymeric material having a first durometer. The second section of the catheter tube is formed of a second polymeric material having a second durometer less than the first durometer. The first section of the catheter tube has a proximal-end portion disposed in a receptacle of the junction and solvent bonded thereto.

In some embodiments, the junction is a tapered distal-end portion of the second section of the catheter tube.

In some embodiments, an abluminal transition between the tapered distal-end portion of the second section of the catheter tube and the proximal-end portion of the first section of the catheter tube is a smooth transition of solvent-interdiffused polymeric material of the first polymeric material and the second polymeric material.

In some embodiments, the junction is a tapered third section of the catheter tube bonded or welded to the second section of the catheter tube. The third section of the catheter tube is formed of a third material having a third durometer less than the first durometer.

In some embodiments, an abluminal transition between the tapered third section of the catheter tube and the proximal-end portion of the first section of the catheter tube is a smooth transition of solvent-interdiffused polymeric material of the first polymeric material and the second polymeric material.

In some embodiments, the RICC is a triluminal catheter. A first lumen of the triluminal catheter terminates at an opening in a distal end of the first section of the catheter tube. A second lumen of the triluminal catheter terminates at a first eyelet in the second section of the catheter tube adjacent the junction. A third lumen of the triluminal catheter terminates at a second eyelet in the second section of the catheter tube adjacent the junction.

In some embodiments, the RICC is a diluminal catheter. A first lumen of the diluminal catheter terminates at an opening in a distal end of the first section of the catheter tube. A second lumen of the diluminal catheter terminates at a first eyelet in the second section of the catheter tube adjacent the junction.

In some embodiments, the first section of the catheter tube is polytetrafluoroethylene, polypropylene, or polyurethane.

In some embodiments, the second section of the catheter tube is polyvinyl chloride, polyethylene, polyurethane, or silicone.

In some embodiments, the RICC has a column strength sufficient to prevent buckling of the catheter tube when inserted into an insertion site and advanced through a vasculature of a patient.

Also disclosed herein is a method of making a RICC including, in some embodiments, an obtaining step of obtaining a first section of a catheter tube formed of a first polymeric material having a first durometer and a second section of the catheter tube formed of a second polymeric material having a second durometer less than the first durometer. The method also includes an applying step of applying solvent to a proximal-end portion of the first section of the catheter tube, a receptacle of a tapered distal-end portion of the second section of the catheter tube, or both the proximal-end portion of the first section of the catheter tube and the receptacle of the tapered distal-end portion of the second section of the catheter tube. The method also includes an inserting step of inserting the proximal-end portion of the first section of the catheter tube into the receptacle of the tapered distal-end portion of the second section of the catheter tube. The method also include an evaporating step of allowing the solvent to evaporate, thereby forming a solvent-bonded junction between the first section of the catheter tube and the second section of the catheter tube.

In some embodiments, the method further includes a tapering step of tapering an untapered distal-end portion of the second section of the catheter tube to form the tapered distal-end portion of the second section of the catheter tube.

In some embodiments, the method further includes a rotating step of rotating the catheter tube along a longitudinal axis thereof. The method also includes an applying step of applying the solvent or a different solvent to an abluminal transition between the proximal-end portion of the first section of the catheter tube and the tapered distal-end portion of the second section of the catheter tube. As a result of the applying step, solvent molecules diffuse into the first polymeric material and the second polymeric material. The method also includes a smoothing step of smoothing the transition with interdiffused polymeric material resulting from entanglement of at least solvated side chains of the first polymeric material and the second polymeric material.

Also disclosed herein is a method of making a RICC including, in some embodiments, an obtaining step of obtaining a first section of a catheter tube formed of a first polymeric material having a first durometer, a second section of the catheter tube formed of a second polymeric material having a second durometer less than the first durometer, and a tapered third section of the catheter tube formed of a third material having a third durometer less than the first durometer. The method also includes an applying step of applying solvent to a proximal-end portion of the first section of the catheter tube, a receptacle of a distal-end portion of the tapered third section of the catheter tube, or both the proximal-end portion of the first section of the catheter tube and the receptacle of the distal-end portion of the tapered third section of the catheter tube. The method also includes an inserting step of inserting the proximal-end portion of the first section of the catheter tube into the receptacle of the distal-end portion of the third section of the catheter tube. The method also includes an evaporating step of allowing the solvent to evaporate, thereby forming a solvent-bonded junction between the first section of the catheter tube and the tapered third section of the catheter tube.

In some embodiments, the method further includes a bonding or welding step of bonding or welding a distal-end portion of the second section of the catheter tube to a proximal-end portion of the tapered third section at or about where a taper of the tapered third section begins.

In some embodiments, the method further includes a rotating step of rotating the catheter tube along a longitudinal axis thereof. The method also includes an applying step of applying the solvent or a different solvent to an abluminal transition between the proximal-end portion of the first section of the catheter tube and the tapered distal-end portion of the third section of the catheter tube. As a result of the applying step, solvent molecules diffuse into the first polymeric material and the third polymeric material. The method also includes a smoothing step of smoothing the transition with interdiffused polymeric material resulting from entanglement of at least solvated side chains of the first polymeric material and the third polymeric material.

Also disclosed herein is a method of using a RICC including, in some embodiments, a creating step of creating an insertion site to access a vasculature of a patient with a needle disposed within a lumen of the RICC. The method also include an inserting step of inserting a distal-end portion of a catheter tube of the RICC into the insertion site. The method also includes an advancing step of advancing the distal-end portion of the catheter tube through the vasculature of the patient without use of a Seldinger technique.

In some embodiments, the method further includes a withdrawing step of withdrawing the needle from the lumen of the RICC after creating the insertion site and inserting at least some of the distal-end portion of the catheter tube into the insertion site.

In some embodiments, the insertion site is at a right subclavian vein or a right internal jugular vein.

In some embodiments, the advancing step includes advancing the distal-end portion of the catheter tube through the right subclavian vein or the right internal jugular vein, a right brachiocephalic vein, and into a superior vena cava.

These and other features of the concepts provided herein will become more apparent to those of skill in the art in view of the accompanying drawings and following description, which describe particular embodiments of such concepts in greater detail.

### DRAWINGS

FIG. 1 illustrates a distal-end portion of a catheter tube of a RICC in accordance with some embodiments.

FIG. 2 illustrates a distal-end portion of a catheter tube of another RICC in accordance with some embodiments.

FIG. 3A illustrates a first transverse cross section of the catheter tube of the RICC of FIG. 1 in accordance with some embodiments.

FIG. 3B illustrates a second transverse cross section of the catheter tube of the RICC of FIG. 1 in accordance with some embodiments.

FIG. 3C illustrates a third transverse cross section of the catheter tube of the RICC of FIG. 1 in accordance with some embodiments.

FIG. 4 illustrates a method of making the RICC of FIG. 1 in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need to reduce the number of steps and medical devices involved in introducing a catheter into a patient and advancing the catheter through a vasculature thereof. Disclosed herein are RICCs and methods thereof that address the foregoing.

### Rapidly Insertable Central Catheters

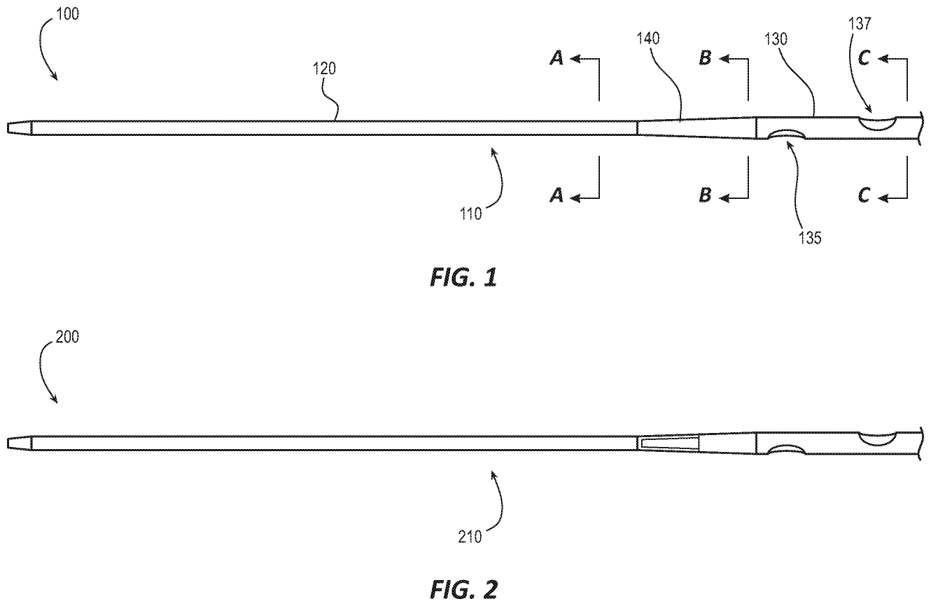

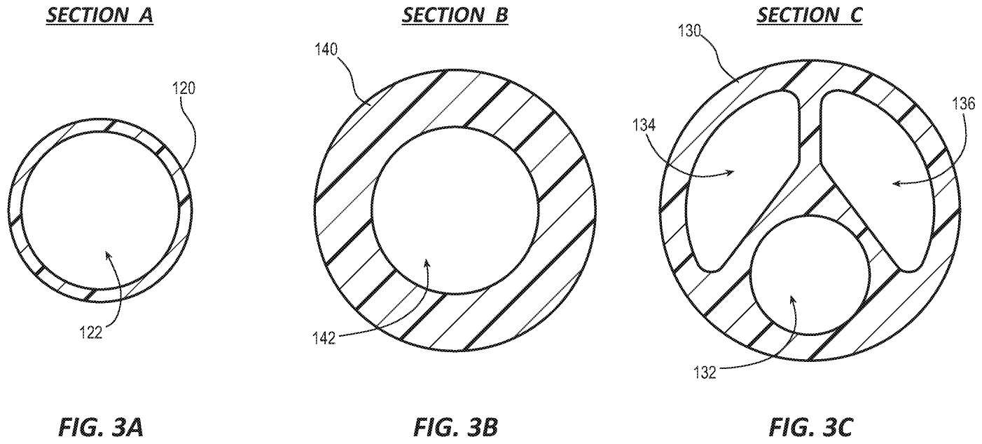

FIG. 1 illustrates a distal-end portion of a catheter tube 110 of a RICC 100 in accordance with some embodiments. FIG. 3A illustrates a transverse cross section of a first section 120 of the catheter tube 110 in accordance with some embodiments. FIG. 3B illustrates a transverse cross section of a second section 130 of the catheter tube 110 in accordance with some embodiments. FIG. 3C illustrates a transverse cross section of a junction 140 of the catheter tube 110 in accordance with some embodiments.

As shown, the RICC 100 includes the first section 120 in the distal-end portion of the catheter tube 110, the second section 130 in the distal-end portion of the catheter tube 110 proximal of the first section 120 of the catheter tube 110, and the junction 140 joining the first section 120 and the second section 130 of the catheter tube 110. The first section 120 of the catheter tube 110 is formed of a first polymeric material having a first durometer. The second section 130 of the catheter tube 110 is formed of a second polymeric material having a second durometer less than the first durometer. The junction 140 can be a third section of the catheter tube 110 joining the first section 120 and the second section 130 of the catheter tube 110 or merely where the first section 120 and the second section 130 of the catheter tube 110 are joined. Together, the first section 120 of the catheter tube 110, the second section 130 of the catheter tube 110, and the junction 140 have a column strength sufficient to prevent buckling of the catheter tube 110 when inserted into an insertion site and advanced through a vasculature of a patient. While the RICC 100 has the foregoing sections, it should be understood other sections and configurations thereof are possible.

The RICC 100 is a triluminal catheter. A first lumen of the triluminal catheter includes fluidly connected luminal sections including a lumen 122 of the first section 120 of the catheter tube 110, a first lumen 132 of the second section 130 of the catheter tube 110, and a lumen 142 of the junction 140. The first lumen terminates at an opening in a distal end of the first section 120 of the catheter tube 110. A second lumen of the triluminal catheter includes a second lumen 134 of the second section 130 of the catheter tube 110. The second lumen 134 terminates at a first eyelet 135 in the second section 130 of the catheter tube 110 adjacent the junction 140. A third lumen of the triluminal catheter includes a third lumen 136 of the second section 130 of the catheter tube 110. The third lumen 136 terminates at a second eyelet 137 in the second section 130 of the catheter tube 110 adjacent the junction 140.

Alternatively, the RICC 100 is a diluminal catheter. Like the foregoing triluminal catheter, a first lumen of the diluminal catheter includes fluidly connected luminal sections including the lumen 122 of the first section 120 of the catheter tube 110, the first lumen 132 of the second section 130 of the catheter tube 110, and the lumen 142 of the junction 140. The first lumen terminates at the opening in a distal end of the first section 120 of the catheter tube 110. A second lumen of the diluminal catheter includes the second lumen 134 of the second section 130 of the catheter tube 110. The second lumen 134 terminates at the first eyelet 135 in the second section 130 of the catheter tube 110 adjacent the junction 140. The third lumen 136 of the second section 130 of the catheter tube 110 is not present in the diluminal catheter.

Further alternatively, the RICC 100 is a monoluminal catheter. Like the foregoing triluminal and diluminal catheters, the lumen of the monoluminal catheter includes fluidly connected luminal sections including the lumen 122 of the first section 120 of the catheter tube 110, the first lumen 132 of the second section 130 of the catheter tube 110, and the lumen 142 of the junction 140. The first lumen terminates at the opening in a distal end of the first section 120 of the catheter tube 110. The second lumen 134 and the third lumen 136 of the second section 130 of the catheter tube 110 is not present in the monoluminal catheter

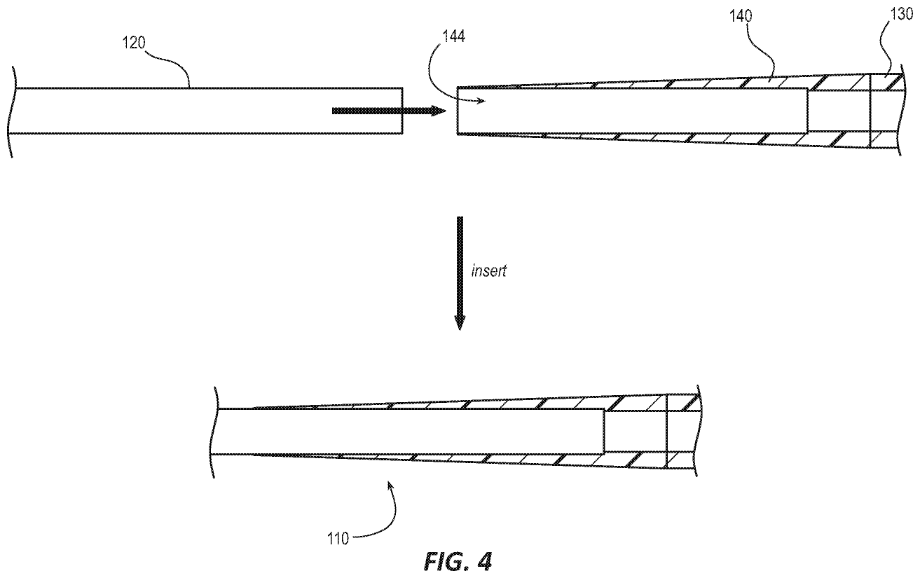

FIG. 4 illustrates a longitudinal cross sections the first section 120 of the catheter tube 110, the second section 130 of the catheter tube 110, and the junction 140 in accordance with some embodiments.

The first section 120 of the catheter tube 110 has a distal-end portion including a tip and a proximal-end portion configured to be disposed in a receptacle 144 of the junction 140 and solvent bonded thereto.

Again, the first section 120 of the catheter tube 110 is formed of a first polymeric material having a first durometer. The first polymeric material can be polytetrafluoroethylene, polypropylene, or polyurethane, but the first polymeric material is not limited to the foregoing polymers. Polyurethane is advantageous in that the first section 120 of the catheter tube 110 can be relatively rigid at room-temperature but become more flexible in vivo at body temperature, which reduces irritation to vessel walls and phlebitis.

The second section 130 of the catheter tube 110 has a distal-end portion optionally including one eyelet or both eyelets of the first eyelet 135 and the second eyelet 137 depending upon whether the catheter is monoluminal, diluminal, or triluminal. While not shown, the second section 130 of the catheter tube 110 also has a proximal-end portion coupled to a hub of the RICC 100.

Again, the second section 130 of the catheter tube 110 is formed of a second polymeric material having a second durometer less than the first durometer of the first polymeric material. The first durometer and the second durometer can be on different scales (e.g., Type A or Type D), so the second durometer might not be numerically less than the first durometer. That said, the hardness of the second polymeric material can still be less than the hardness of the first polymeric material as the different scales—each of which ranges from 0 to 100—are designed for characterizing different materials in groups of the materials having a like hardness. The second polymeric material can be polyvinyl chloride, polyethylene, polyurethane, or silicone, but the first polymeric material is not limited to the foregoing polymers. Polyurethane is advantageous in that can be less thrombogenic than some other polymers.

Notwithstanding the foregoing, the first section 120 and the second section 130 of the catheter tube 110 can be formed of a same polymeric material or different polymeric materials having substantially equal durometers provided a column strength of the catheter tube 110 is sufficient to prevent buckling of the catheter tube 110 when inserted into an insertion site and advanced through a vasculature of a patient.

The junction 140 can be a third section of the catheter tube 110. The junction 140 includes a tapered distal-end portion as well as a proximal-end portion that is either untapered or includes a taper at or about where the taper of the tapered distal-end portion of the junction 140 begins. The proximal-end portion of the junction 140 abuts the distal-end portion of the second section 130 of the catheter tube 110 in a solvent bond or a heat weld.

The junction 140 is formed of a third polymeric material having a third durometer less the first durometer of the first polymeric material of the first section 120 of the catheter tube 110. Like that set forth above, such durometers can be on different scales (e.g., Type A or Type D), so the third durometer might not be numerically less than the first durometer. Alternatively, the third polymeric material can have a substantially equal durometer to that of the first polymeric material of the first section 120 of the catheter tube 110 or a durometer more than the first polymeric material of the first section 120 of the catheter tube 110—provided a column strength of the catheter tube 110 is sufficient to prevent buckling of the catheter tube 110 when inserted into an insertion site and advanced through a vasculature of a patient. The third polymeric material can have a substantially equal durometer to that of the second polymeric material of the second section 130 of the catheter tube 110 or a different durometer than the second polymeric material of the second section 130 of the catheter tube 110 such as a durometer more than the second polymeric material of the second section 130 of the catheter tube 110. As set forth herein, different durometers can be used provided a column strength of the catheter tube 110 is sufficient to prevent buckling of the catheter tube 110 when inserted into an insertion site and advanced through a vasculature of a patient.

As an alternative to the foregoing, the second section 130 of the catheter tube 110 includes the junction 140 or the third section of the catheter tube 110. That is, the third section of the catheter tube 110 is not formed separately from the second section 130 of the catheter tube 110 and bonded or welded thereto—but integrally with the second section 130 of the catheter tube 110. Like that set forth above for the junction 140, the second section 130 of the catheter tube 110 includes a tapered distal-end portion when the second section 130 of the catheter tube 110 includes the junction 140 or the third section of the catheter tube 110. Polymeric materials for the second section 130 are set forth above.

An abluminal transition between the proximal-end portion of the first section 120 of the catheter tube 110 and the tapered distal-end portion of the junction 140 is a smooth transition of solvent-interdiffused polymeric material of the first polymeric material and the third polymeric material. Alternatively, if the second section 130 of the catheter tube 110 is integral with the junction 140 or the third section of the catheter tube 110, an abluminal transition between the proximal-end portion of the first section 120 of the catheter tube 110 and the second section 130 of the catheter tube 110 is a smooth transition of solvent-interdiffused polymeric material of the first polymeric material and the second polymeric material. The foregoing abluminal transition is best shown in the bottom of FIG. 4. “Smooth” in the context of either one of the foregoing smooth transitions indicates the abluminal transition between the first section 120 of the catheter tube 110 and the tapered distal-end portion of the junction 140 includes a sufficiently small to negligible edge that precludes catching skin when the RICC 100 is inserted into an insertion site of a patient.

FIG. 2 illustrates a distal-end portion of a catheter tube 210 of another RICC 200 in accordance with some embodiments. The RICC 200 and methods thereof are detailed in U.S. application Ser. No. 17/006,553, filed Aug. 28, 2020, and in U.S. application Ser. No. 17/031,478, filed Sep. 24, 2020, each of which is incorporated by reference in its entirety into this application.

### Methods

FIG. 4 illustrates an inserting step of a method for making the RICC 100 of FIG. 1 in accordance with some embodiments. A first method for making the RICC 100 including the inserting step of FIG. 4 is directed to forming the catheter tube 110 from the first section 120 of the catheter tube 110, the second section 130 of the catheter tube 110, and the junction 140. A second method for making the RICC 100 including the inserting step of FIG. 4 is directed to forming the catheter tube 110 from the first section 120 of the catheter tube 110 and the second section 130 of the catheter tube 110, wherein the second section 130 of the catheter tube 110 includes the junction 140 or the third section of the catheter tube 110.

The first method for making the RICC 100 includes an obtaining step of obtaining the first section 120 of the catheter tube 110 formed of the first polymeric material having the first durometer, the second section 130 of the catheter tube 110 formed of the second polymeric material having the second durometer less than the first durometer, and the third section of the catheter tube 110 or the junction 140 formed of the third material having the third durometer less than the first durometer. The junction 140 includes the tapered distal-end portion thereof as set forth above.

The first method also includes an applying step of applying solvent to the proximal-end portion of the first section 120 of the catheter tube 110, the receptacle 144 of the distal-end portion of the junction 140, or both the proximal-end portion of the first section 120 of the catheter tube 110 and the receptacle 144 of the distal-end portion of the junction 140.

As shown in FIG. 4, the first method also includes an inserting step of inserting the proximal-end portion of the first section 120 of the catheter tube 110 into the receptacle 144 of the distal-end portion of the junction 140.

The first method also includes an evaporating step of allowing the solvent to evaporate, thereby forming a solvent-bonded junction between the first section 120 of the catheter tube 110 and the junction 140.

The first method can also include a bonding or welding step of bonding or welding the distal-end portion of the second section 130 of the catheter tube 110 to the proximal-end portion of the junction 140 at or about where the taper of the tapered distal-end portion of the junction 140 begins.

The first method can also include a rotating step of rotating the catheter tube 110 along a longitudinal axis thereof.

The first method can also include an applying step of applying the solvent or a different solvent to an abluminal transition between the proximal-end portion of the first section 120 of the catheter tube 110 and the tapered distal-end portion of the junction 140. As a result of the applying step, solvent molecules diffuse into the first polymeric material and the third polymeric material.

The first method can also include a smoothing step of smoothing the abluminal transition with interdiffused polymeric material resulting from entanglement of at least solvated side chains of the first polymeric material and the third polymeric material.

The second method for making the RICC 100 includes an obtaining step of obtaining the first section 120 of a catheter tube 110 formed of the first polymeric material having the first durometer and the second section 130 of the catheter tube 110 formed of the second polymeric material having the second durometer less than the first durometer.

The second method can also include a tapering step of tapering an untapered distal-end portion of the second section 130 of the catheter tube 110 to form the tapered distal-end portion of the second section 130 of the catheter tube 110.

The second method also includes an applying step of applying solvent to the proximal-end portion of the first section 120 of the catheter tube 110, the receptacle 144 of the tapered distal-end portion of the second section 130 of the catheter tube 110, or both the proximal-end portion of the first section 120 of the catheter tube 110 and the receptacle 144 of the tapered distal-end portion of the second section 130 of the catheter tube 110.

As shown in FIG. 4, the second method also includes an inserting step of inserting the proximal-end portion of the first section 120 of the catheter tube 110 into the receptacle 144 of the tapered distal-end portion of the second section 130 of the catheter tube 110.

The second method also includes an evaporating step of allowing the solvent to evaporate, thereby forming a solvent-bonded junction between the first section 120 of the catheter tube 110 and the second section 130 of the catheter tube 110.

The second method can also include a rotating step of rotating the catheter tube 110 along a longitudinal axis thereof.

The second method can also include an applying step of applying the solvent or a different solvent to an abluminal transition between the proximal-end portion of the first section 120 of the catheter tube 110 and the tapered distal-end portion of the second section 130 of the catheter tube 110. As a result of the applying step, solvent molecules diffuse into the first polymeric material and the second polymeric material.

The second method can also include a smoothing step of smoothing the abluminal transition with interdiffused polymeric material resulting from entanglement of at least solvated side chains of the first polymeric material and the second polymeric material.

A method of using the RICC 100 includes a creating step of creating an insertion site to access a vasculature of a patient with a needle disposed within a lumen of the RICC 100. The insertion site can be at a subclavian vein such as a right or left subclavian vein, an internal jugular vein such as a right or left internal jugular vein, or a femoral vein.

The method also includes an inserting step of inserting the distal-end portion of the catheter tube 110 of the RICC 100 into the insertion site.

The method also includes a withdrawing step of withdrawing the needle from the lumen of the RICC 100 after creating the insertion site and inserting at least some of the distal-end portion of the catheter tube 110 into the insertion site.

The method also includes an advancing step of advancing the distal-end portion of the catheter tube 110 through the vasculature of the patient without use of the Seldinger technique. For example, if the insertion site is at the right subclavian vein or the right internal jugular vein, the advancing step can include advancing the distal-end portion of the catheter tube 110 through the right subclavian vein or the right internal jugular vein, a right brachiocephalic vein, and into a superior vena cava. Other insertions sites such as at the left subclavian vein or the left internal jugular vein require advancing the distal-end portion of the catheter tube 110 through corresponding vasculature.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A rapidly insertable central catheter (“RICC”), comprising:
a first section of a catheter tube formed of a first polymeric material having a first durometer, the first section in a distal-end portion of the catheter tube;
a second section of the catheter tube formed of a second polymeric material having a second durometer less than the first durometer, the second section in the distal-end portion of the catheter tube proximal of the first section; and
a junction joining the first and second sections of the catheter tube, the first section of the catheter tube having a proximal-end portion disposed in a receptacle of the junction and solvent bonded thereto.

2. The RICC of claim 1, wherein the junction is a tapered distal-end portion of the second section of the catheter tube.

3. The RICC of claim 2, wherein an abluminal transition between the tapered distal-end portion of the second section of the catheter tube and the proximal-end portion of the first section of the catheter tube is a smooth transition of solvent-interdiffused polymeric material of the first polymeric material and the second polymeric material.

4. The RICC of claim 1, wherein the junction is a tapered third section of the catheter tube bonded or welded to the second section of the catheter tube, the third section of the catheter tube formed of a third material having a third durometer less than the first durometer.

5. The RICC of claim 4, wherein an abluminal transition between the tapered third section of the catheter tube and the proximal-end portion of the first section of the catheter tube is a smooth transition of solvent-interdiffused polymeric material of the first polymeric material and the second polymeric material.

6. The RICC of claim 1, wherein the RICC is a triluminal catheter having a first lumen terminating at an opening in a distal end of the first section of the catheter tube, a second lumen terminating at a first eyelet in the second section of the catheter tube adjacent the junction, and a third lumen terminating at a second eyelet in the second section of the catheter tube adjacent the junction.

7. The RICC of claim 1, wherein the RICC is a diluminal catheter having a first lumen terminating at an opening in a distal end of the first section of the catheter tube and a second lumen terminating at a first eyelet in the second section of the catheter tube adjacent the junction.

8. The RICC of claim 1, wherein the first section of the catheter tube is polytetrafluoroethylene, polypropylene, or polyurethane.

9. The RICC of claim 1, wherein the second section of the catheter tube is polyvinyl chloride, polyethylene, polyurethane, or silicone.

10. The RICC of claim 1, wherein the RICC has a column strength sufficient to prevent buckling of the catheter tube when inserted into an insertion site and advanced through a vasculature of a patient.

11. A method of making a rapidly insertable central catheter (“RICC”), comprising:
obtaining a first section of a catheter tube formed of a first polymeric material having a first durometer and a second section of the catheter tube formed of a second polymeric material having a second durometer less than the first durometer;
applying solvent to a proximal-end portion of the first section of the catheter tube, a receptacle of a tapered distal-end portion of the second section of the catheter tube, or both the proximal-end portion of the first section of the catheter tube and the receptacle of the tapered distal-end portion of the second section of the catheter tube;
inserting the proximal-end portion of the first section of the catheter tube into the receptacle of the tapered distal-end portion of the second section of the catheter tube; and
allowing the solvent to evaporate, thereby forming a solvent-bonded junction between the first section of the catheter tube and the second section of the catheter tube.

12. The method of claim 11, further comprising tapering an untapered distal-end portion of the second section of the catheter tube to form the tapered distal-end portion of the second section of the catheter tube.

13. The method of claim 11, further comprising:
rotating the catheter tube along a longitudinal axis thereof; and
applying the solvent or a different solvent to an abluminal transition between the proximal-end portion of the first section of the catheter tube and the tapered distal-end portion of the second section of the catheter tube, thereby diffusing solvent molecules into the first polymeric material and the second polymeric material; and
smoothing the transition with interdiffused polymeric material resulting from entanglement of at least solvated side chains of the first polymeric material and the second polymeric material.

14. A method of making a rapidly insertable central catheter (“RICC”), comprising:
obtaining a first section of a catheter tube formed of a first polymeric material having a first durometer, a second section of the catheter tube formed of a second polymeric material having a second durometer less than the first durometer, and a tapered third section of the catheter tube formed of a third material having a third durometer less than the first durometer;
applying solvent to a proximal-end portion of the first section of the catheter tube, a receptacle of a distal-end portion of the tapered third section of the catheter tube, or both the proximal-end portion of the first section of the catheter tube and the receptacle of the distal-end portion of the tapered third section of the catheter tube;
inserting the proximal-end portion of the first section of the catheter tube into the receptacle of the distal-end portion of the third section of the catheter tube; and
allowing the solvent to evaporate, thereby forming a solvent-bonded junction between the first section of the catheter tube and the tapered third section of the catheter tube.

15. The method of claim 14, further comprising bonding or welding a distal-end portion of the second section of the catheter tube to a proximal-end portion of the tapered third section at or about where a taper of the tapered third section begins.

16. The method of claim 14, further comprising:
rotating the catheter tube along a longitudinal axis thereof; and
applying the solvent or a different solvent to an abluminal transition between the proximal-end portion of the first section of the catheter tube and the tapered distal-end portion of the third section of the catheter tube, thereby diffusing solvent molecules into the first polymeric material and the third polymeric material; and
smoothing the transition with interdiffused polymeric material resulting from entanglement of at least solvated side chains of the first polymeric material and the third polymeric material.

17. A method of a rapidly inserted central catheter (“RICC”), comprising:
creating an insertion site to access a vasculature of a patient with a needle disposed within a lumen of the RICC;
inserting a distal-end portion of a catheter tube of the RICC into the insertion site; and
advancing the distal-end portion of the catheter tube through the vasculature of the patient without use of a Seldinger technique.

18. The method of claim 17, further comprising withdrawing the needle from the lumen of the RICC after creating the insertion site and inserting at least some of the distal-end portion of the catheter tube into the insertion site.

19. The method of claim 17, wherein the insertion site is at a right subclavian vein or a right internal jugular vein.

20. The method of claim 19, wherein advancing the distal-end portion of the catheter tube through the vasculature of the patient includes advancing the distal-end portion of the catheter tube through the right subclavian vein or the right internal jugular vein, a right brachiocephalic vein, and into a superior vena cava.
